(19) United States
(12) Patent Application Publication
(10) Pub. No.: US 2025/0318937 A1
(43) Pub. Date: Oct. 16, 2025

(54) SURGICAL INSTRUMENTS, SURGICAL INSTRUMENT SYSTEMS AND METHODS OF SURGERY

(71) Applicant: DePuy Ireland Unlimited Company, Ringaskiddy (IE)

(72) Inventors: Alasdair Mercer, Leeds (GB); James Brooks, Burley in Wharfedale (GB); Michael Rock, Leeds (GB)

(21) Appl. No.: 19/175,317

(22) Filed: Apr. 10, 2025

(30) Foreign Application Priority Data
Apr. 12, 2024 (GB) 2405198.9

(51) Int. Cl. A61F 2/46

(52) U.S. Cl. CPC A61F 2/4603; A61F 2002/4622

(57) ABSTRACT

A surgical instrument for positioning and orienting instruments for use in an orthopaedic surgical knee procedure is disclosed. A surgical instrument system and method of performing an orthopaedic surgical knee procedure are also disclosed.

This application claims priority to Great Britain Patent Application Serial No. 2405198.9 which was filed on Apr. 12, 2024, and is hereby incorporated by reference in its entirety.

### TECHNICAL FIELD

This disclosure is concerned with improvements in and relating to surgical instruments, such as clamping instruments, surgical instrument systems, such as systems including clamping instruments, and methods of surgery, such as methods including the use of clamping instruments. The instruments and other aspects are particularly applicable to orthopaedic surgery, including surgery on the knee joint.

### BACKGROUND

In a variety of surgical methods, correct positioning and orientation of surgical instruments relative to the surgical site is important both for the conduct of the surgery and for the subsequent successful outcome for the patient. This includes orthopaedic surgery, such as knee joint procedures including those where prosthetic implants are provided for the joint.

For instance, incorrect alignment of a tibial jig instrument can result in the angles for the implant being off and so giving uneven loading in use.

Unfortunately, this alignment is best checked both from the front (anterior) and side (lateral), but that may involve impermissible movement of the surgeon outside the sterile zone of the surgery and so the alignment is conventionally checked as far as possible from within the sterile zone. This anterior view can cause errors in alignment due to parallax and other factors, such as referencing the tibial jig to the axis of the tibia covered in skin and muscle tissue.

### SUMMARY

According to a first aspect of the disclosure there is provided a surgical instrument, the instrument comprising: an elongate element having a distal portion and a proximal portion and extending in a proximal-distal direction; a clamp operating mechanism mounted on the elongate element; two or more clamp elements, connected to the distal portion of the elongate element and controlled by the clamp operating mechanism, the two or more clamp elements having a first open state and a second engaging state; a mounting element, connected to the proximal portion of the elongate element, the mounting element having a first position in the proximal-distal direction and a second position in the proximal-distal direction, the first position being further from the two or more clamp elements than the second position; and wherein that the clamp operating mechanism is operable to move the two or more clamp elements between the first open state and the second engaging state, the two or more clamp elements having a mid-point that remains constant between the first open state and the second engaging state, and the mounting element and the mid-point between the clamp elements define a longitudinal axis that is configured to be positioned co-linear with a tibial mechanical axis of a patient during use.

The elongate element may have a variable proximal-distal direction length, for instance a greater proximal-distal extent to provide the first position for the mounting element and/or a lesser proximal-distal extent to provide the second position for the mounting element.

The mounting element and/or a depending element extending there from, may in use, be spaced from a first anatomical feature of a patient, particularly proximally, in the first position. The mounting element and/or a depending element extending there from, may in use, abut a first anatomical feature of a patient in the second position. The mounting element and/or particularly a depending element extending there from, may in use, penetrate a first anatomical feature of a patient in the second position.

The mounting element and/or depending element extending therefrom in the first position may oppose a portion of a tibia, particularly a portion of a proximal end of the tibia, such as a portion of a proximal facing surface of the tibia. The mounting element and/or depending element extending therefrom in the first position may oppose a portion of an ACL tibial insertion footprint and/or a portion of a notch intermediate a medial intercondylar tubercule and a lateral intercondylar tubercule.

The mounting element and/or depending element extending therefrom in the second position may abut or penetrate a portion of a tibia, particularly a portion of a proximal end of the tibia, such as a portion of a proximal facing surface of the tibia. The mounting element and/or depending element extending therefrom in the second position may abut or penetrate a portion of a an ACL tibial insertion footprint and/or a portion of a notch intermediate a medial intercondylar tubercule and a lateral intercondylar tubercule.

The mounting element and/or depending element extending therefrom may resist rotation of the instrument about the proximal-distal axis. The mounting element or more particularly the depending element extending therefrom may be a pointed element, such as a spike or a pin. The mounting element may have a plurality of depending elements extending therefrom. A plurality of spikes may extend therefrom. One spike may extend further distally than another spike. A plurality of pins may extend therefrom. One pin may extend further distally than another pin.

The mounting element and/or depending element extending therefrom may move distally during transition from the first position to the second position. The mounting element and/or depending element extending therefrom may move proximally during transition from the second position to the first position. The mounting element and/or depending element extending therefrom may not move posteriorly-anteriorly during transition from the first position to the second position. The mounting element and/or depending element extending therefrom may not move posteriorly-anteriorly during transition from the second position to the first position. The mounting element and/or depending element extending therefrom may not move medially-laterally during transition from the first position to the second position. The mounting element and/or depending element extending therefrom may not move medially-laterally during transition from the second position to the first position.

The mounting element and/or depending element extending therefrom may abut or penetrate the bone at the proximal exit of the tibial axis from the tibia. Where a plurality of mounting elements are used, the longer and/or the first mounting element may abut or penetrate the bone at the proximal exit of the tibial axis from the tibia.

The first open state for the two or more clamping elements, in use, may allow one or more clamp elements to be introduced to one side of a second anatomical feature of a patient, potentially with the first open state also allowing with one or more other clamp elements to be introduced to a second side of a further anatomical feature of a patient. The first side and the second side may be two sides of the same anatomical feature. The second anatomical feature and the further anatomical feature may be the same feature.

The second anatomical feature and the further anatomical feature may be different from the first anatomical feature.

The second engaging state for the two or more clamping elements, in use, may prevent one or more clamp elements being removed from one side of an anatomical feature of a patient, potentially with the second engaging state also preventing one or more other clamp elements being removed from a second side of an anatomical feature of a patient. The first side and the second side may be two sides of the same anatomical feature.

The two or more clamping elements in the engaging state may, in use, engage with an ankle area of the patient.

One or more of the clamping elements in the engaging state may, in use, engage with a malleolus, such as a medial malleolus, particularly a medial malleolus of the tibia. A further one or more of the clamping elements in the engaging state may, in use, engage with a malleolus, for instance another malleolus, such as a lateral malleolus, particularly a lateral malleolus of the fibula of the patient. The two or more clamping elements in the engaging state may, in use, engage with a medial malleolus of the tibia and a lateral malleolus of the fibula of the patient.

One or more of the clamping elements in the engaging state may, in use, engage with a medial extremity of the medial malleolus, particularly of the tibia. A further one or more of the clamping elements in the engaging state may, in use, engage with a lateral extremity of the lateral malleolus of the patient, particularly of the fibula. The two or more clamping elements in the engaging state may, in use, engage with a medial extremity of the medial malleolus and a lateral extremity of the lateral malleolus of the patient. The two or more clamping elements in the engaging state may, in use, engage with a medial extremity of the medial malleolus of the tibia and a lateral extremity of the lateral malleolus of the fibula of the patient.

The first clamp elements may oppose the second clamp elements in the open state and/or closed state and/or clamping state.

The anatomy data relating to a set of patients may define a length across one malleolus to another malleolus. The length may be defined between the medial-most part of the medial malleolus and the lateral-most part of the lateral malleolus. The medial-most and the lateral-most parts may be the extremities, the extremity M^{ext} of the medial malleolus and the extremity L^{ext} of the lateral malleolus. The length may be the distance between extremity M^{ext} of the medial malleolus and the extremity L^{ext} of the lateral malleolus. The two or more clamping elements may have a separation distance between them, particularly at or towards a posterior end. In the open state the separation distance may be greater than the length. In the engaging state, the separation distance may be less than or equal to the length. The separation distance may be measured medially-laterally and/or may be measure in the same plane as the length.

Two or more or all of the clamping elements may have equivalent shapes and configurations. Two or more of the clamping elements, on opposing sides to each other, may be mirror images of each other.

One or more or all of the clamping elements may include a posterior extending element. One or more or all of the clamping elements may include a posterior end portion. The posterior extending portion may have a lower profile than the posterior end portion. The posterior end portion may provide a clamping location. The clamping location may be provided on a surface, for instance an opposing surface, of the clamping element. The surface may be recessed within the posterior end portion.

The surface may be recessed within the posterior end portion, with the extent of recessing increasing from the posterior end towards the anterior end. The recessing may increase to a maximum depth. From the maximum depth location, the recessing may decrease from the maximum depth towards the anterior end.

The recessing of the surface may increase from the proximal side towards the distal side. The recessing may increase to a, for instance the, maximum depth. From a maximum depth location, the recessing may decrease from the maximum depth towards the distal side. The surface may define a dished recess, particularly with the clamping location therein, for instance at a or the maximum depth location.

The recessing of the surface may increase from the proximal side towards the distal side. The recessing may increase to a, for instance the, maximum depth. From a maximum depth location, the recessing may decrease from the maximum depth towards the distal side. The surface may define a dished recess, particularly with the clamping location therein, for instance at a or the maximum depth location.

The recessing of the surface may be consistent from the posterior end towards the anterior end and/or from the anterior end towards the posterior end. The maximum depth may extend from towards the posterior end towards the anterior end with the same maximum depth. The maximum depth may extend from the posterior end towards the anterior end with the same maximum depth. The maximum depth may be midway between the proximal and distal sides of the posterior end portion.

The elongate element may have a variable proximal-distal direction length, for instance a greater proximal-distal extent to provide the first position for the mounting element and/or a lesser proximal-distal extent to provide the second position for the mounting element, by the elongate element being telescopic. The elongate element may comprise a first part and a second part, the second part being seated within the first part and being slidable therein. The second part may be more proximal than the first part. A releasable position locking unit may be provided, for instance a clamp, potentially provided on the first element, such as a collar element.

For alternative or additional proximal-distal adjustment, extension and/or retraction of the first distal extending element may be provided, for instance relative to an offset element. The first distal extending element may have a variable proximal-distal direction length. The first distal extending element may be provided with a first part and a second part, the second part being seated within the first part and being slidable therein. The second part may be more distal that the first part. The first distal extending part may be variable in length in a proximal-distal direction, for instance parallel to but spaced from the elongate element. The first distal extending element may be fixed in extent in a medial-lateral direction and/or anterior-posterior direction. A releasable position locking unit may be provided, for instance a clamp. Alternatively, or additionally, proximal-distal adjustment, extension and/or retraction of the second distal extending element may be provided, for instance relative to an offset element. The second distal extending element may have a variable proximal-distal direction length. The second distal extending element may be provided with a first part and a second part, the second part being seated within the first part and being slidable therein. The second part may be more distal that the first part. The second distal extending part may be variable in length in a proximal-distal direction, for instance parallel to but spaced from the elongate element. A releasable position locking unit may be provided, for instance a clamp. The second distal extending element may be fixed in extent in a medial-lateral direction and/or anterior-posterior direction.

The variability in length could be provided by the elongate element alone. The variability could be provided by the first distal extending element and/or second distal extending element. The variability could be provided by a combination of the elongate element and/or the first distal extending element and/or second distal extending element.

The variability in length may provide a variable length of 200 mm to 500 mm, for instance 220 mm to 480 mm, such as 240 mm to 460 mm. The variable length may be measured between an intersection between the elongate element and a second elongate element and an intersection between the elongate element and one or more clamping elements. The variable length may be measured between an intersection between the instrument and a plane extending from the proximal tibia surface and intersection between the instrument and a plane extending from the lateral or medial malleolus. The variability in length, for instance the telescopic arrangement, may provide a variation in the range of at least 300 mm, for instance at least 260 mm, such as at least 220 mm.

The mounting element may be connected to the proximal portion of the elongate element by a second elongate element. The second elongate element may extend in the anterior-posterior direction. The second elongate element may have a variable anterior-posterior length. The second elongate element may be provided with a first part and a second part, the second part being seated within the first part and being slidable therein. The second part may be more posterior than the first part. The second elongate element may be variable in length in an anterior-posterior direction, for instance parallel to but spaced from the clamping elements. A releasable position locking unit may be provided, for instance a clamp.

The variability in length, for instance the telescopic arrangement, may provide a variable length of 30 mm to 120 mm, for instance 30 mm to 100 m, such as 40 mm to 90 mm. The variability in length, for instance the telescopic arrangement, may provide a variation of at least 40 mm, for instance at least 50 mm, such as at least 60 mm, potentially at least 75 mm. The variability in length, for instance the telescopic arrangement, may provide a variation of less than 100 mm, for instance less than 80 mm, such as less than 70 mm, potentially less than 60 mm.

The variability in length provides a separation between the elongate element and the tibia for a cutting block. The variability in length may be considered as the length between the elongate element and the mounting element, particularly the depending element extending therefrom. The variability in length may be considered as the length between the elongate element and the ACL tibial insertion footprint. The second elongate element may be fixed in extent in a medial-lateral direction and/or variable in extent in the proximal-distal direction, particularly through variation of the extent of the elongate element.

One or more or all of the clamping elements may extend in the anterior-posterior direction. One or more or all of the clamping elements may have a variable anterior-posterior length. One or more or all of the clamping elements may be provided with a first part and a second part, the second part being seated within the first part and being slidable therein. The second part may be more posterior than the first part. One or more or all of the clamping elements may be variable in length in an anterior-posterior direction, for instance parallel to but spaced from the second elongate element. One or more or all of the clamping elements may be variable in extent in a proximal-distal direction, particularly through variation of the extent of the elongate element. A releasable position locking unit may be provided, for instance a clamp. One or more or all of the clamping elements may be variable in extent in a medial-lateral direction, particularly due to transition of the clamping arms between the open state and the engaging state and/or between the engaging state and the open state.

The variability in length, for instance the telescopic arrangement, may provide a variable length of 30 mm to 120 mm, for instance 30 mm to 100 m, such as 40 mm to 90 mm. The variability in length, for instance the telescopic arrangement, may provide a variation of at least 40 mm, for instance at least 50 mm, such as at least 60 mm, potentially at least 75 mm. The variability in length, for instance the telescopic arrangement, may provide a variation of less than 100 mm, for instance less than 80 mm, such as less than 70 mm, potentially less than 60 mm. The variability in length may be considered as the length between the elongate element and the posterior end[s] of the one or more clamping elements, particularly the maximum depth location thereon. The variability in length may be considered as the length between the elongate element and one or both of the malleoli.

Where the length of an element is variable, such as any one or more or all of the elongate element, the second elongate element, the clamping element[s], the distal depending element[s], then an indication of the length of that element may be provided. The indication of length may be a scale, for instance with one or more indicia representing a value for the element. Where a desired length for each of two or more elements is sought, the indicia may have the same value when those two or more elements have the intended lengths. The desired length may be the same or a known and desired relative length. The scale and/or indicia may be provided on the element whose length is to be indicated.

The location where a part of the patient abuts a clamping element may be defined as the clamping location. An indicator may be provided for the position of the clamping position in the anterior-posterior direction and/or distal-proximal direction and/or medial-lateral direction. The indicator may be provided for the position of the clamping location, where the clamping location is not constrained or defined in a given direction by the profile of the clamping element[s]. The indication may particularly relate to the posterior-anterior direction. The indicator may include a scale of positions and/or an indicium and/or value representing the present position. The indicator may be positioned by the surgeon to correspond with the clamping location. The indicator may be provided extending along a proximal edge of the clamping element, particularly a posterior end portion thereof. The indicia and/or value may be used in the setting of the position of one or more other elements which are variable.

The clamp operating mechanism mounted on the elongate element may include a first element which can moved on the elongate element, for instance a sliding movement. The first element may provide a first pivotal mounting. The clamp operating mechanism may include a first pivotal mounting provided on a collar element.

The clamp operating mechanism mounted on the elongate element may include a second element which is fixed relative to, for instance on, the elongate element. The second element may provide a second pivotal mounting. The first element and the second element may be spaced apart in the proximal-distal direction. The clamp operating mechanism may include a second pivotal mounting provided on a boss element. Both the collar element and the boss element may be provided on the elongate element, potentially spaced apart in the proximal/distal direction.

A first pair of operating arms may be pivotally connected by a first pivot at their first end to the first pivotal mounting, for instance on the collar element. A second pair of operating arms may be pivotally connected by a second pivot at their first end to the second pivotal mounting, for instance on the boss element. The second end of one of the first pair of operating arms and the second end of one of the second pair of operating arms may be pivotally connect to one another by a third pivot. The second end of the other of the first operating arms and the second end of the other of the second pair of operating arms may be pivotally connected to one another by a fourth pivot.

In an embodiment, one of the proximal operating arms may be connected to a first offset element, for instance a first proximal offset element, such as a collar. One of the distal operating arms may be connected to a first offset element, for instance a first distal offset element, such as a collar. Both one of the proximal operating arms may be connected to a first proximal offset element and one of the distal operating arms may be connected to a first distal offset element. The another of the proximal operating arms may be connected to a second off set element, for instance a second proximal offset element, such as a collar. The another of the distal operating arms may be connected to a second offset element, for instance a second distal offset element. Both the another of the proximal operating arms may be connected to a second proximal offset element and the another of the distal operating arms may be connected to a second distal offset element.

In an alternative embodiment, the third pivot may be connected to a first offset element. The fourth pivot may be connected to a second offset element.

The first element, such as a collar element, may be adapted to slide proximally and distally on the elongate element. A stop may define the distal-most position for the collar element. In the distal-most position, or there towards, the open state for the instrument may be provided and/or the separation of the third pivot and the fourth pivot is at a maximum and/or the separation of the first offset element and second offset element is at a maximum. A second stop may define a proximal-most position for the collar element. In the proximal-most position, a closed state for the instrument may be provided and/or the separation of the first offset element and second offset element is at a minimum. Intermediate the open state and the closed state is an engaging or clamping state wherein the clamping elements abut the patient, for instance the medial malleolus of the tibia and the lateral malleolus of the fibula of the patient.

The first pair of operating arms may extend distally from the first element towards the third pivot and towards the fourth pivot. The second pair of operating arms may extend proximally from the second element towards the third pivot and towards the fourth pivot. The first pair and second pair of operating arms may define a diamond shape and/or parallelogram. The arms of the first pair of arms may be the same length as the arms of the second pair of arms.

In another embodiment, the first pair of operating arms may extend distally from the first element towards the third pivot and towards the fourth pivot. The second pair of operating arms may extend distally from the second element towards the third pivot and towards the fourth pivot. The first pair and second pair of operating arms may each define an inverted V shape. The arms of the first pair of arms may be longer than the arms of the second pair of arms.

The first operating arm and the second operating arm on a first side of the elongate element may have a different length to the first operating arm and the second operating arm on a second side of the elongate element. The first side may be the medial side. The second side may be the lateral side. The first operating arm and the second operating arm on a first side of the elongate element may have a different length, such that the clamping element[s] to the first side move less during transition than the clamping element[s] to the second side. The first operating arm and the second operating arm on a first side of the elongate element may have a different length, such that the tibial mechanical axis is defined as other than at the 50:50 mid-point relative to the two malleolus limits. The first operating arm and the second operating arm on a first side of the elongate element may have a different length, such that 55:45 split on distance between the two malleolus limits as assumed by the elongate element of the instrument and/or by the main axis of the instrument. The first operating arm and the second operating arm on a first side of the elongate element may have a different length, the different lengths controlling the movement inward and outward on the first side and on the second side.

Where the first operating arm and the second operating arm on a first side of the elongate element have a different length to the first operating arm and the second operating arm on a second side of the elongate element, the instrument may be provided with an indicator of orientation and/or of side to which the different values of movement apply, for instance which is the 55 and which is the 45 side in the 55:45 split of movement. The indicator or orientation and/or of side may indicate tibia side and/or or fibula side on the different sides. The indicator or orientation and/or of side may indicate left leg and right leg on different faces of the instrument.

The first offset element may be provided with a first distal extending element. The second offset element may be provided with a second distal extending element. The first and second distal extending elements may extend parallel to the elongate element, potentially axially spaced from the elongate element.

In an embodiment, a first distally extending element may be provided by a first proximal offset element, such as a collar. A first distally extending element may be provided by a first distal offset element, such as a collar. A first distally extending element may be provided by a first proximal offset element and a first distal offset element. A first distally extending element may be provided extending through a first proximal offset element and/or a first distal offset element.

The first distally extending element may be slidably received in a first proximal offset element and/or a first distal offset element. The first distally extending element may have a clamped and a released state in a first proximal offset element or a first distal offset element.

A first proximal offset element and/or a first distal offset element may be pivotally mounted. A first proximal offset element may be pivotally mounted on one of the proximal operating arms. A first distal offset element may be pivotally mounted on one of the distal operating arms. Both the first proximal offset element and the first distal offset element may be so mounted.

In the embodiment, a second distally extending element may be provided by a second proximal offset element, such as a collar. A second distally extending element may be provided by a second distal offset element, such as a collar. A second distally extending element may be provided by a second proximal offset element and a second distal offset element. A second distally extending element may be provided extending through a second proximal offset element and/or a second distal offset element.

The second distally extending element may be slidably received in a second proximal offset element and/or a second distal offset element. The second distally extending element may have a clamped and a released state in a second proximal offset element or a second distal offset element.

A second proximal offset element and/or a second distal offset element may be pivotally mounted. A second proximal offset element may be pivotally mounted on one of the proximal operating arms. A second distal offset element may be pivotally mounted on one of the distal operating arms. Both the second proximal offset element and the second distal offset element may be so mounted.

Particularly at a distal end, the first distal extending element may be provided with one or more first clamp elements. Particularly at a distal end, the second distal extending element may be provided with one or more second clamp elements.

The instrument may be provided with one or more further attachment mechanisms. The instrument may be provided with a further attachment mechanism by a strapping mechanism. For instance, an elongate member may be connected to a first location on the instrument and an elongate member retainer may be provided at a second location of the instrument. In use, the elongate member may be extended out and around a part of the patient and then taken to the elongate member retainer, for instance to be engaged therewith. The elongate member may be a strap. The elongate member may be a spring. The elongate member may contact the patient at a secondary retaining location. The secondary retaining location may be around the ankle or around the foot or around the tibia and fibula.

Any feature or combination of features referenced as being a part of the intermediate component can be provided on the surgical component and/or the further surgical component as an alternative. Any feature or combination of features referenced as being a part of the surgical instrument can be provided on the intermediate component as an alternative. Any combination of features described as provided on the surgical instrument and intermediate component can be provided in the reverse configuration with the surgical instrument feature[s] being transferred to the intermediate component and the intermediate component feature[s] being transferred to the surgical instrument.

The first aspect of the invention may include any of the features, options or possibilities set out elsewhere in this application, including in the other aspects and the description.

According to a second aspect of the disclosure there is provided a surgical instrument system, the instrument comprising: a first surgical instrument, a second surgical instrument for releasable connection to the first instrument; the first surgical instrument comprising: an elongate element having a distal portion and a proximal portion and extending in a proximal-distal direction; a clamp operating mechanism mounted on the elongate element; two or more clamp elements, connected to the distal portion of the elongate element and controlled by the clamp operating mechanism, the two or more clamp elements having a first open state and a second engaging state; a mounting element, connected to the proximal portion of the elongate element, the mounting element having a first position in the proximal-distal direction and a second position in the proximal-distal direction, the first position being further from the two or more clamp elements than the second position; and wherein that the clamp operating mechanism is operable to move the two or more clamp elements between the first open state and the second engaging state, the two or more clamp elements having a mid-point that remains constant between the first open state and the second engaging state, and the mounting element and the mid-point between the clamp elements define a longitudinal axis that is configured to be positioned co-linear with a tibial mechanical axis of a patient during use.

The first surgical instrument may include any of the features, options or possibilities set out elsewhere in this application, including in the other aspects and the description for the instrument. The second surgical instrument may be a further component for the first instrument, such as an adjustment component for mounting on the first instrument.

The adjustment component may include a connection mechanism for mounting the adjustment component on the first instrument. The connection mechanism may provide a proximal mounting and/or a distal mounting.

The proximal mounting may be provided on a proximal part of the elongate element of the first instrument cooperating with the adjustment component. The proximal part cooperating may vary in position. The cooperation may be provided by the elongate element passing through a proximal aperture in the adjustment component. The elongate element may be slidably provided in the proximal aperture. The aperture may be defined by a collar connected to a support.

The proximal mounting may include a pivotal mounting. The pivotal mounting may be provided between a collar and a support. The pivotal mounting may be provided between a collar and a bracket element provided on the support, for instance at the proximal end of the support.

The pivotal mounting may extend in a posterior-anterior direction. The pivotal mounting may allow for rotation of the adjustment component relative to the first instrument. The pivotal mounting may allow for rotation of the bracket element and/or support and/or slot relative to the elongate element.

The proximal mounting may include a second pivotal mounting. The second pivotal mounting may be provided between the support and a frame. The second pivotal mounting may be provided between a bracket element and a frame. The second pivotal mounting may be provided by one or more bracket arms extending from the bracket element, for instance on a lateral and/or medial side of the elongate element.

The second pivotal mounting may extend in a lateral-medial direction. The second pivotal mounting may allow for rotation of the adjustment component relative to the first instrument. The second pivotal mounting may allow for rotation of the frame relative to the first instrument. A locking mechanism may be provided to prevent rotation from a selected position.

The frame may be connected to the second pivotal mounting towards the proximal end of the frame. The frame may extend proximally beyond the second pivotal mounting to a proximal end. A mounting element for a third instrument may be provided at or towards the proximal end of the frame, particularly more proximally than the second pivotal mounting. The third instrument may be one or more cutting blocks.

The distal mounting may be provided by a distal part of the elongate element of the first instrument cooperating with the adjustment component. The distal part cooperating may vary in position. The cooperation may be provided by the elongate element passing through a distal slot in the adjustment component. The slot may be defined in a support of the adjustment component. The slot may be defined by a first posterior member and a second anterior member. The posterior member and anterior member may have opposing walls with a consistent spacing between them, for instance corresponding to the dimension of the elongate element in an anterior-posterior direction. The slot may extend in a medial-lateral direction. The slot may allow for rotation of one or more other parts of the adjustment component. The slot may allow the elongate element to slide distally and/or proximally. A locking mechanism may be provided to prevent rotation from a selected position.

The support, for instance the second anterior element, may include one or more scales, such as an indicia-based scale, for instance to measure the rotation of the adjustment component. A scale may be provided to one side and a second scale may be provided to the other side. The first scale may include a value for a Varus adjustment. The second scale may provide a value for a Valgus adjustment.

A support may be provided connecting the proximal mounting to the distal mounting. The support may have a stem extending distally-proximally. The proximal mounting may be provided on the proximal end of the stem. The distal mounting may be provided on the distal end of the stem. The first posterior element may extend medially and laterally from the stem. The second anterior element may extend medially and laterally from the stem. A projection may extend anteriorly from the support, for instance from the second anterior element.

A projection may provide support for a section of the frame, for instance a distal end of the frame. The projection may include a distal surface. The distal surface may have an arcuate profile, for instance the arc that the distal end of the frame rotates through. The projection may include one or more scales, such as an indicia-based scale, for instance to measure the rotation of the frame, potentially indicating the tibial slope angle. A scale may be provided on one or both lateral-medial sides of the projection.

The system may include a third instrument. The third instrument may be connectable to the frame, for instance to a mounting element provided towards or at the proximal end of the frame.

The third instrument may be one or more cutting blocks. One or more or all of the cutting blocks may be provided with a cutting tool slot. Two or more cutting blocks may be provided with different cutting slots, for instance with different angles. The cutting block may include a male element or a female element. The mounting element may include the other of a male element or a female element. The male element may engage in use with a female element.

Any feature or combination of features referenced as being a part of the intermediate component can be provided on the surgical component and/or the further surgical component as an alternative. Any feature or combination of features referenced as being a part of the surgical instrument can be provided on the intermediate component as an alternative. Any combination of features described as provided on the surgical instrument and intermediate component can be provided in the reverse configuration with the surgical instrument feature[s] being transferred to the intermediate component and the intermediate component feature[s] being transferred to the surgical instrument.

The second aspect of the invention may include any of the features, options or possibilities set out elsewhere in this application, including in the other aspects and the description.

According to a third aspect of the disclosure there is provided a surgical method, the method comprising: providing an instrument, the instrument comprising: an elongate element having a distal portion and a proximal portion and extending in a proximal-distal direction; a clamp operating mechanism mounted on the elongate element; two or more clamp elements, connected to the distal portion of the elongate element and controlled by the clamp operating mechanism, the two or more clamp elements having a first open state a second engaging state, and; a mounting element, connected to the proximal portion of the elongate element, the mounting element having a first position in the proximal-distal direction and a second position in the proximal-distal direction, the first position being further from the two or more clamp elements than the second position; providing the two or more clamping elements in a first open state; exposing the proximal end of the tibia of a patient at a surgical site; abutting the mounting element or an element depending therefrom with the proximal end of the tibia within the surgical site; introducing the two or more clamping elements to the ankle region of the patient; transitioning the two or more clamping elements from the first open state to the second engaging state to engage the clamping elements with the ankle region of the patient such that the mounting element and the mid-point between the clamp elements define a longitudinal axis that is co-linear with a tibial mechanical axis of the patient.

The method may include a mounting element and/or a depending element extending there from being spaced from a first anatomical feature of a patient, particularly proximally, in the first position. The mounting element and/or a depending element extending there from may be moved to abut a first anatomical feature of a patient in the second position. The mounting element and/or particularly a depending element extending there from may be moved to penetrate a first anatomical feature of a patient in the second position.

The method may include the mounting element and/or depending element extending therefrom may be moved to the first position opposing a portion of a tibia, particularly a portion of a proximal end of the tibia, such as a portion of a proximal facing surface of the tibia. The mounting element and/or depending element extending therefrom may be moved to the first position opposing a portion of a medial intercondylar tubercule, or a portion of a lateral intercondylar tubercule or a portion of a notch, for instance a portion of a notch intermediate a medial intercondylar tubercule and a lateral intercondylar tubercule.

The method may include the mounting element and/or depending element extending therefrom being moved to the second position to abut or penetrate a portion of a tibia, particularly a portion of a proximal end of the tibia, such as a portion of a proximal facing surface of the tibia. The mounting element and/or depending element extending therefrom may be moved to the second position to abut or penetrate a portion of a medial intercondylar tubercule, or a portion of a lateral intercondylar tubercule or a portion of a notch, for instance a portion of a notch intermediate a medial intercondylar tubercule and a lateral intercondylar tubercule.

The method may include the mounting element and/or depending element extending therefrom being moved distally during transition from the first position to the second position. The mounting element and/or depending element extending therefrom may be moved proximally during transition from the second position to the first position.

In the first open state for the two or more clamping elements, one or more clamp elements may be introduced to one side of a second anatomical feature of a patient, potentially with one or more other clamp elements to be introduced to a second side of a further anatomical feature of a patient. The first side and the second side may be two sides of the same anatomical feature.

In the second engaging state for the two or more clamping elements, the engagement may prevent one or more clamp elements being removed from one side of an anatomical feature of a patient, potentially with the second engaging state also preventing one or more other clamp elements being removed from a second side of an anatomical feature of a patient.

The two or more clamping elements in the engaging state may, in use, engage with an ankle area of the patient.

One or more of the clamping elements in the engaging state may engage with a malleolus, such as a medial malleolus, particularly a medial malleolus of the tibia. A further one or more of the clamping elements in the engaging state may engage with a malleolus, for instance another malleolus, such as a lateral malleolus, particularly a lateral malleolus of the fibula of the patient. The two or more clamping elements in the engaging state may engage with a medial malleolus of the tibia and a lateral malleolus of the fibula of the patient.

One or more of the clamping elements in the engaging state may engage with a medial extremity of the medial malleolus, particularly of the tibia. A further one or more of the clamping elements in the engaging state may engage with a lateral extremity of the lateral malleolus of the patient, particularly of the fibula. The two or more clamping elements in the engaging state may engage with a medial extremity of the medial malleolus and a lateral extremity of the lateral malleolus of the patient. The two or more clamping elements in the engaging state may engage with a medial extremity of the medial malleolus of the tibia and a lateral extremity of the lateral malleolus of the fibula of the patient.

The ankle region may be clamped with no surgical sites provided in the ankle region. The engaging state is provided with the skin over the malleolus.

In the open state, a separation distance may be provided between the posterior end portions, and this may be greater than the medial to lateral extent of any ankle joint. This may allow the clamping elements to be advanced posteriorly with one clamping element to one side of the ankle joint and the other clamping element on the other side of the ankle joint.

A recess on the clamping element may be generally aligned with the position of the medial malleolus. A recess on the clamping element may be generally aligned with the position of the lateral malleolus. This may be provided in the open state or during transition from the open state towards the engaged state.

The method may include reducing the separation distance and/or positioning the clamping elements to abut the medial malleolus and the lateral malleolus. The positioning may provide them within the recess on their respective clamping element. The method may include further reduce the separation distance to bring that distance down to the length of the medial to lateral extent of the ankle joint 380, for instance the length between the extremity M^{ext} of the medial malleolus and the extremity L^{ext} of the lateral malleolus. The clamping elements, particularly the recesses and, for instance, the clamping locations, may abut the ankle joint, particularly the medial malleolus and the lateral malleolus. The abutment may be with the extremity M^{ext} of the medial malleolus and the extremity L^{ext} of the lateral malleolus.

The method may include, potentially with the engaging state provided at the ankle, an adjustment to increase the length of the elongate element. The adjustment may be made to give a proximal/distal extent sought, for instance to allow the mounting element to be moved over the proximal end surface of the tibia.

The method may include, potentially with the engaging state provided at the ankle, an adjustment to reduce the length of the elongate element. The adjustment may be made to give a proximal/distal extent sought, for instance to abut the mounting element, or a depending element extending therefrom, with the proximal end surface of the tibia.

The method may include the depending element extending therefrom penetrating the bone of the patient, for instance to give the second position.

A further releasable clamp may be used to maintain the length of the elongate element for either the first position and/or the second position.

The method may include providing the instrument in alignment with the tibia. The alignment may be with the tibial mechanical axis, potentially determined as extending from a proximal location of the tibia, potentially with reference to an ACL tibial insertion footprint and/or a portion of a notch intermediate a medial intercondylar tubercule and a lateral intercondylar tubercule, to a distal location of the tibia, potentially determined as a mid-point between the medial malleolus and the lateral malleolus.

The method may provide an alignment check step, for instance on whether this alignment is achieved or not. The instrument, including the clamping elements and mounting element should provide the alignment, but adjustment may be provided.

The surgical method may include, for instance after an alignment check step, one or more further surgical steps.

The method may include a second surgical instrument, such as a further component for the first instrument, for instance an adjustment component for mounting on the first instrument.

The method may include the selection and/or introduction of a further component to the instrument, for instance with the further component being an adjustment component.

The method may include the selection and/or introduction of a tibial cutting block, for instance to the adjustment component.

The method may include rotating the second instrument, for instance an adjustment component, relative to the first instrument, for instance a clamping component. The method may include rotation about a first axis and/or about a second axis.

The first axis may be a posterior-anterior extending axis. The method may include an adjustment for Valgus and/or Varus, for instance a rotational adjustment.

The second axis may be a medial-lateral extending axis. The method may include an adjustment for tibia slope, for instance a rotational adjustment.

The method may include resection of the tibia and/or femur. The method may include trialling of a prosthetic implant. The method may include providing and/or fixing a prosthetic implant.

Any feature or combination of features referenced as being a part of the intermediate component can be provided on the surgical component and/or the further surgical component as an alternative. Any feature or combination of features referenced as being a part of the surgical instrument can be provided on the intermediate component as an alternative. Any combination of features described as provided on the surgical instrument and intermediate component can be provided in the reverse configuration with the surgical instrument feature[s] being transferred to the intermediate component and the intermediate component feature[s] being transferred to the surgical instrument.

The third aspect of the invention may include any of the features, options or possibilities set out elsewhere in this application, including in the other aspects and the description.

### BRIEF DESCRIPTION OF THE DRAWINGS

Various embodiments of the disclosure will now be described, by way of example only, and with reference to the accompanying Figures, in which:

FIG. 1a is an anterior view of a left lower leg with respect to the knee joint, fibula and tibia;

FIG. 1b is an anterior view of the left lower leg with respect to the ankle joint;

FIG. 1c is a view of the proximal surface of a tibia;

FIG. 1d is a perspective offset view of a tibia and fibula showing relevant anatomical planes and slopes;

FIG. 2a is a prior art clamp component and cooperating adjustment component of a tibial jig;

FIG. 2b shows the clamp component and the adjustment component assembled to provide the tibial jig of FIG. 2a assembled;

FIG. 2c shows the tibial jig and a cooperating tibial cutting block;

FIG. 3a is a first embodiment of an instrument according to the disclosure;

FIG. 3b is a plan view of the clamping mechanism of FIG. 3a in an open state;

FIG. 3c is a plan view of the clamping mechanism of FIG. 3b in a closed state;

FIG. 3d is a view of the first embodiment of the instrument detailing an adjustment component of the instrument connected thereto;;

FIG. 3e is a view of the first embodiment detailing the clamping mechanism and clamping arm interaction;

FIG. 4a is a detailed view of a first alternative embodiment of a mounting element;

FIG. 4b is a detailed view of a second alternative embodiment of a mounting element;

FIG. 5a is a second embodiment of an instrument according to the disclosure;

FIG. 5b is a detailed view of the cooperation of the clamping elements of the instrument of FIG. 5a with the ankle area;

FIG. 6 is a third embodiment of an instrument according to the disclosure with a split ratio of relative movement of the clamping elements; and

FIG. 7 is a fourth embodiment of an instrument according to the disclosure with additional clamping.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

Terms representing anatomical references, such as anterior, posterior, medial, lateral, superior, inferior, etcetera, may be used throughout the specification in reference to the orthopaedic implants or prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

In a variety of surgical methods, correct positioning and orientation of surgical instruments relative to the surgical site is important both for the conduct of the surgery and for the subsequent successful outcome for the patient. This includes orthopaedic surgery, such as knee joint procedures including those where prosthetic implants are provided for the joint.

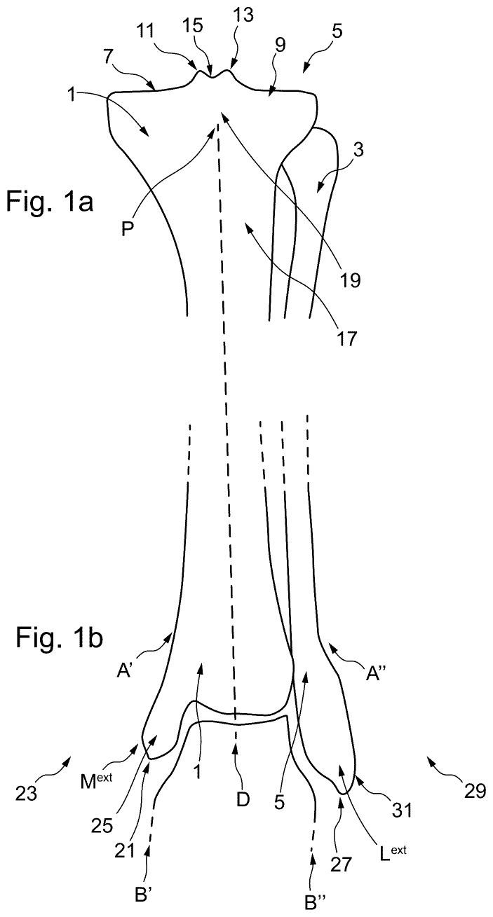

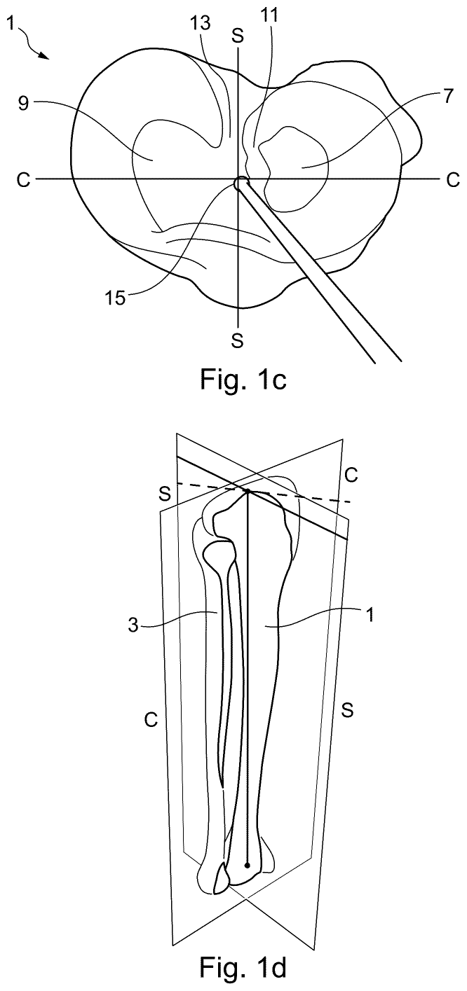

With reference to FIG. 1a, the anatomy illustrated includes a tibia 1 and fibula 3. This is an anterior view of a left leg.

At the proximal end 5, the tibia 1 is profiled with a first curved articular surface 7 and a second curved articular surface 9. These cooperate with the condyles of the femur. Between the first curved articular surface 7 and the second curved articular surface 9 there is the raised area formed of the medial intercondylar tubercule 11, the lateral intercondylar tubercule 13 and an ACL tibial insertion footprint 15, a notch often, there between. On the anterior surface 17 of the tibia 1 is a ridge defined by the tibial tubercule anterior continuation 19. The tibial tubercule anterior continuation 19 is part way between the first curved articular surface 7 and the second curved articular surface 9.

The distal end 21 of the tibia 1 is shown in more detail in FIG. 1b. On the medial side 23, is the medial malleolus 25. At the distal end 27 of the fibula 3, on the lateral side 29, is the lateral malleolus 31.

In the view of the proximal surface of the tibia 1 seen in FIG. 1c, the first curved articular surface 7 and a second curved articular surface 9, together with the medial intercondylar tubercule 11, the lateral intercondylar tubercule 13 and an ACL tibial insertion footprint 15 are all visible. Overlain on the view are a representation of the Sagital plane, S-S, and the Coronal plane, C-C. The extent and orientation of these planes are also clearly shown in the right leg of FIG. 1d. The intersection between the Sagital plane, S-S, and the Coronal plane, C-C, defines the tibial mechanical axis P-D.

During surgery, the tibial mechanical axis P-D is considered to extend from a proximal location P, often defined with reference to the ACL tibial insertion footprint 15, to a distal location D, often determined as a mid-point 33 between the medial malleolus 25 and the lateral malleolus 31.

During surgery, it is important to align the tibial jig used with the tibial mechanical axis P-D. To do so in manually controlled surgical procedures, the proximal location P and the distal location D are established from visual inspection and then the tibial mechanical axis P-D is established between them. Whether the tibial jig is aligned therewith, is then established by the eye of the surgeon. It is important to the success of the surgical procedure, that the proximal-distal extending tibial jig is parallel to the tibial mechanical axis P-D. Incorrect alignment of the tibial jig can result in the angles for the implant being off and so giving uneven loading in use. Unfortunately, this alignment is best checked from the front [anterior] and side [lateral], but that may involve impermissible movement of the surgeon outside the sterile zone of the surgery and so the alignment is conventionally checked as far as possible from within the sterile zone. This anterior view can cause errors in alignment due to parallax and other factors, such as referencing the tibial jig to the axis of the tibia covered in skin and muscle issue.

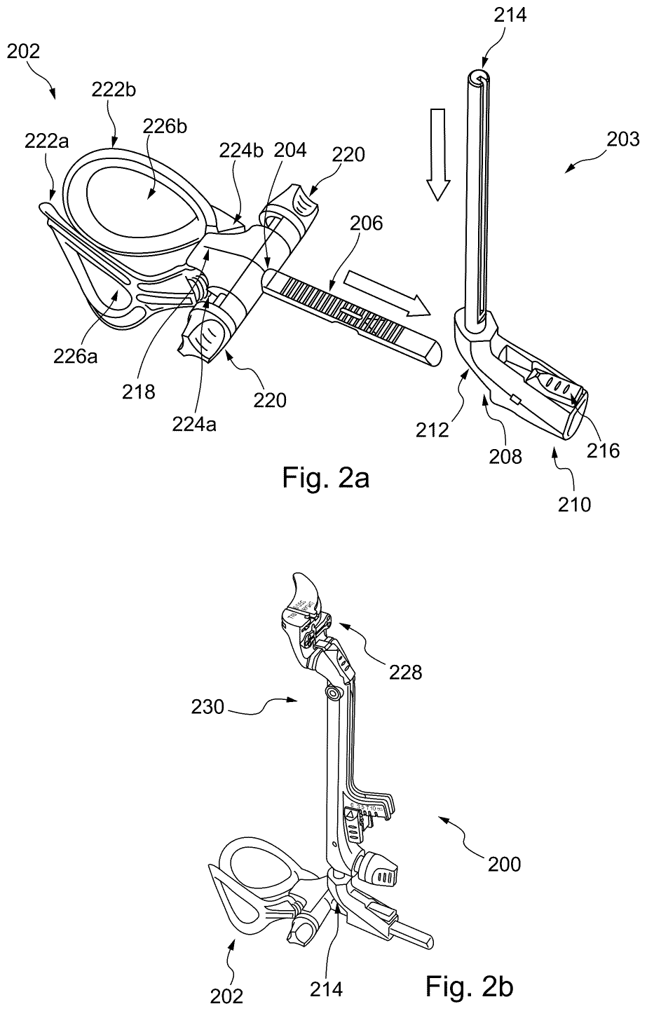

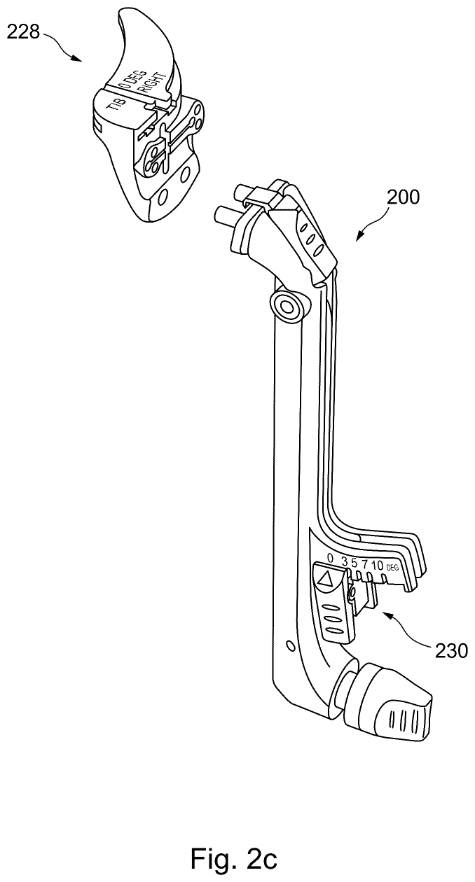

For manually controlled operations, an example of an existing approach to the provision and alignment of a tibial jig instrument 200 using an ankle clamp component 202, distal up-rod component 203 and proximal adjustment component 230 is shown in FIGS. 2a, 2b and 2c.

Referring to FIG. 2a, the ankle clamp 202 is provided with a body element 204 from which a ratchet 206 extends. The ratchet 206 slides into a slot 208 in an adjustment body 210 provided at the distal end 212 of an extramedullary tibial distal up-rod 214; overall forming the up-rod component 203. The ratchet 206 and slot 208 engagement is controlled by a button 216 to allow the spacing of the up-rod 214 relative to the body element 204 of the ankle clamp 202 to be adjusted.

The body element 204 provides an axis of lateral-medial movement aligned with the long axis of the ratchet 206. This allows side to side movement of the ratchet 206, by rotating the knobs 220, and hence movement of the up-rod 214 relative to the clamp element 218. This controls the Varus/Vagus adjustment for the up-rod 214 and hence the adjustment component 230. The adjustment is a lead screw so that rotating the knobs 220 causes element 204 to move along a medial/lateral axis.

The clamp element 218 provides a mount for a pair of opposing clamp arms 222a, 222b. Springs 224a, 224b bias the opposing clamp arms 222a, 222b towards one another.

To mount the ankle clamp 202 in the ankle area of a patient, the opposing clamp arms 222a, 222b are manually opened and placed either side of the ankle. The opposing clamp arms 222a, 222b then close around the ankle under the force applied by the springs 224a, 224b. The opposing clamp arms 222a, 222b each have a large central cut out 226a, 226b such that the medial malleolus 25 and the lateral malleolus 31 sit in the cut outs 226a, 226b and are not contacted by the opposing clamp arms 222a, 222b.

In terms of this prior art clamping action, referring to FIG. 1b, the abutment of the opposing clamp arms 222a, 222b are at locations A′, A″ B′ and B″. These locations, particularly locations A, and A″ are also typical of other clamping actions, such as belts or springs passes around the leg or clamps with pairs of opposing bar like arms. None these approaches provide clamping which contacts and sits on the malleoli.

In practice, when the ankle clamp 202 is mounted on the patient, the adjustment component 230 and the up-rod component 203 is already provided on the up-rod 214 [as shown in FIG. 2b] and the tibial cutting block 228 is already provided on the adjustment component 230.

With the ankle clamp 202 in-situ, the adjustment component 230 thereon is adjusted. The details of a adjustment component 230 can be seen in FIG. 2c. The posterior slope adjustment mechanism 232 is used to control the angle of the adjustment component 230 relative to the tibia 1 and is set appropriately for the implant configuration being used.

The Varus/Vagus adjustment sought, through the side to side movement is controlled using the knobs 220, as described above.

The height adjustment knob 234 is used to retain and release the adjustment component 230 relative to the up-rod 214 to allow adjustment in the proximal/distal directions.

The instruments of the present disclosure provide an improved approach to the determination of the datum for the correct centring and alignment of the instrument, such as a tibial jig instrument.

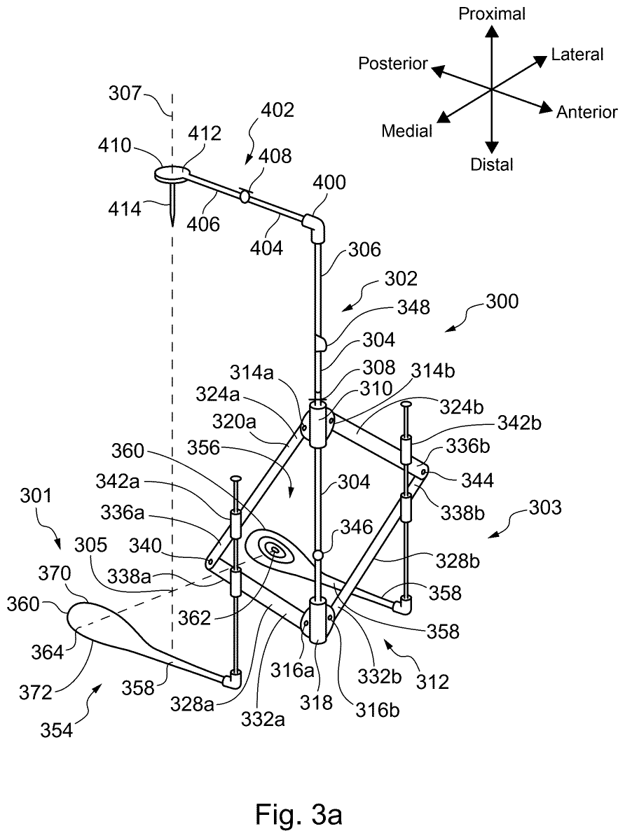

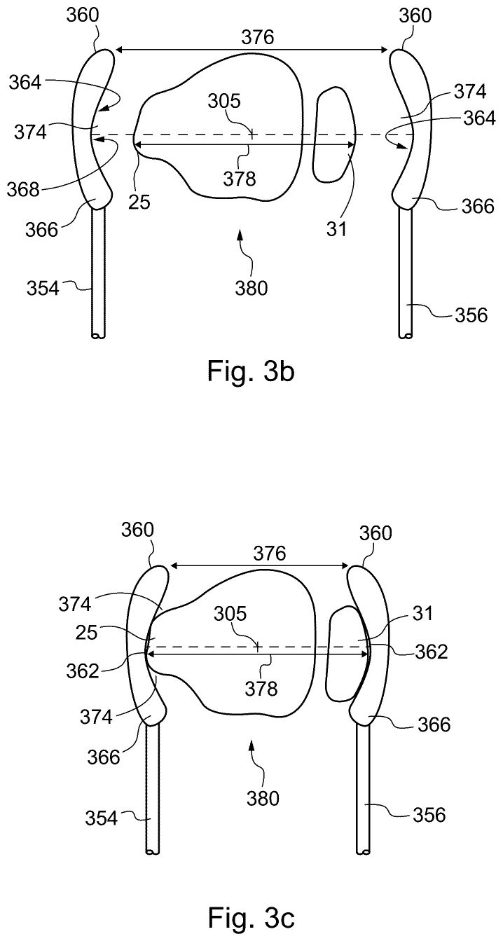

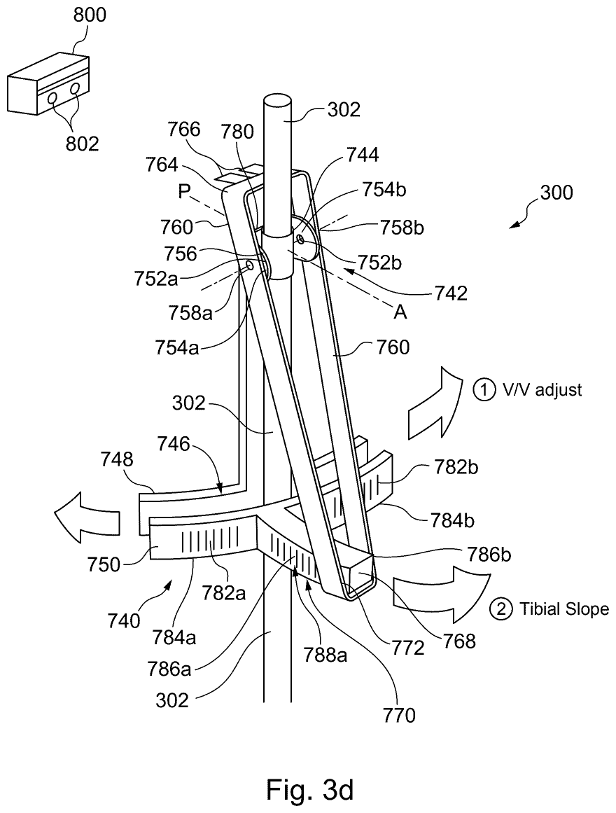

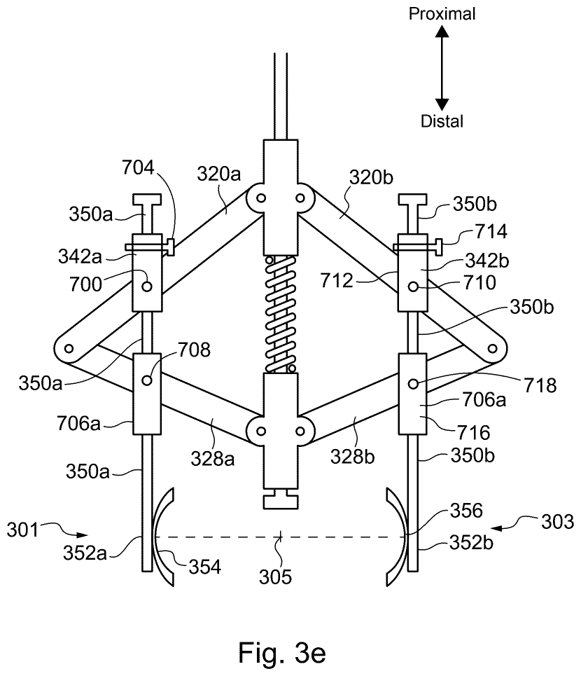

The surgical instrument 300, as illustrated in the FIG. 3a-3d, is a first embodiment of the disclosure. Alongside the embodiment of the instrument 300 the surgical directions typically applying during use are also shown; the proximal-distal direction; the medial-lateral direction [reversed for the other leg] and the posterior-anterior direction.

The surgical instrument 300 is a clamping instrument including a medial paddle 301 configured to abut the medial malleolus 25 and a lateral paddle 303 configured to abut the lateral malleolus 31 of the patient. As described in greater detail below, the surgical instrument 300 includes a clamp operating mechanism 312 that is configured to move the paddles 301, 303 between an open state in which they can be positioned over the patient's ankle and any of a number of clamping states in which the paddles 301, 303 engage the ankle of any patient. As shown in FIG. 3c, the clamp operating mechanism 312 is operable to position the paddles 301, 303 over the ankle such that a mid-point 305 between the paddles 301, 303 remains aligned with the mid-point between the medial malleolus 25 and the lateral malleolus 31 (and therefore with the distal location D of the tibial mechanical axis P-D) at any of the clamping states, regardless of the size of the patient's ankle.

As described in greater detail below, the surgical instrument 300 also includes a proximal mounting element 412, which is configured to abut the ACL tibial insertion footprint 15 (and therefore the proximal location P of the tibial mechanical axis P-D). A longitudinal axis 307 is defined by the proximal mounting element 412 and the mid-point 305 between the paddles 301, 303, which is positioned to be co-linear with the tibial mechanical axis P-D of the patient during use, thereby properly positioning the surgical instrument 300 for the proximal resection.

The instrument 300 is provided with an elongate element 302 which in use extends in the proximal/distal directions. The length of this elongate element 302 in those directions can be adjusted as the elongate element 302 is formed of a first part 304 and a second part 306 seated within the first part 304 and able to telescope into and out of the first part 304. A releasable clamp 308 is provided to fix the overall length of the elongate element 302 at the desired extent. Typically, the telescopic arrangement seeks to provide a variable length range of 240 mm to 460 mm.

A clamp operating mechanism 312 is provided mounted on the elongate element 302. In this embodiment, the clamping operating mechanism 302 is formed of two first pivotal mountings 314a, 314b provided on the collar element 310 and two second pivotal mounting 316a, 316b provided on a boss element 318. Both the collar element 310 and the boss element 318 are provided on the elongate element 302 and are spaced apart in the proximal/distal direction. A first pair of operating arms 320a, 320b are pivotally connected by a first pivotal mounting 314a, 314b at their first end 324a, 324b to the collar element 310.

A second pair of operating arms 328a, 328b are pivotally connected by the second pivotal mountings 316a, 316b at their first end 332a, 332b. The second end 336a of one 320a of the first pair of operating arms and the second end 338a of one 328a of the second pair of operating arms are pivotally connect to one another by a third pivot 340. The second end 336b of the other 320b of the first operating arms and the second end 338b of the other 328b of the second pair of operating arms are pivotally connected to one another by a fourth pivot 344.

The collar element 310 is able to slide proximally and distally on the elongate element 302. A stop 346 defines the distal-most position for the collar element 310, as shown in FIG. 3a. In this position, an open state for the instrument, the separation of the third pivot 340 and the fourth pivot 344 is at a maximum, as is the separation of the first offset element 342a and second offset element 342b. If the collar element 310 moves in a proximal direction, then the separation reduces. A proximal-most position is controlled by a second stop 348. In this position, the closed state for the instrument, the separation of the first offset element 342a and second offset element 342b is at a minimum. In use, intermediate the open state and the closed state is a clamping state where the clamping locations, described further below, abut the medial malleolus 25 and the lateral malleolus 31 of the patient.

Referring to FIGS. 3a and 3e, the mounting of the medial paddle 301 and the lateral paddle 303 are shown. It should be appreciated that the terms “medial paddle” and “lateral paddle” are relative to the patient's anatomy and, depending on the leg of the patient, the surgical instrument 300 may be positioned such that the medial paddle is on the lateral side of the patient's ankle (and is therefore the lateral paddle) and the lateral paddle is on the medial side of the patient's ankle (and is therefore the medial paddle) in such uses. The medial paddle 301 includes a first proximal offset element 342a, which is mounted on one arm 320a of the first pair of arms by a first proximal pivot 700. The first proximal offset element 342a includes a proximal sleeve 702 which receives a first distal extending element 350a. The first distal extending element 350a can slide within the proximal sleeve 702, but can also be held and maintained in position, in a distal-proximal sense [for instance by a clamp 704]. Even when held in position the first distal extending element 350a is free to rotate relative to the one arm 320a of the first pair of arms, such that the first distal extending element 350a remains aligned distal-proximal direction as the arms move and remains parallel to the second distal extending element 350b.

The first distal extending element 350a also extends through a distal sleeve provided by a first distal offset element 706a, with the first distal offset element 706a mounted on one arm 328a of the second pair of arms by a first distal pivot 708. The first distal extending element 350a can slide within the distal sleeve and, as the first distal extending element 350a is free to rotate relative to the one arm 328a of the second pair of arms too, the first distal extending element 350a remains aligned in the same distal-proximal direction as the arms 326a, 328a move, and remains parallel to a second distal extending element 350b. As shown in FIG. 3a, the medial paddle 301 further includes a clamping element 354 that is coupled to the distal extending element 350a.

On the other side of the clamping mechanism, the lateral paddle 303 includes a second proximal offset element 342b, which is mounted on one arm 320b of the first pair of arms by a first proximal pivot 710. The second proximal offset element 342b includes a proximal sleeve 712, which receives a second distal extending element 350b. The second distal extending element 350b can slide within the proximal sleeve 712, but can also be held and maintained in position, in a distal-proximal sense [for instance by a clamp 714]. Even when held in position the second distal extending element 350b is free to rotate relative to the one arm 320b of the first pair of arms, such that the second distal extending element 350b remains aligned in a distal-proximal direction as the arms move and remains parallel to the second distal extending element 350b.

The second distal extending element 350b also extends through a distal sleeve 716 provided by a second distal offset element 706a, with the second distal offset element 706a mounted on one arm 328b of the second pair of arms by a first distal pivot 718. The second distal extending element 350b can slide within the distal sleeve 716 and, as the second distal extending element 350b is free to rotate relative to the one arm 328b of the second pair of arms too, the second distal extending element 350b remains aligned in the same distal-proximal direction as the arms 326b, 328b move, and remains parallel to the first distal extending element 350a. As shown in FIG. 3e, the lateral paddle 303 further includes a clamping element 356 that is coupled to the distal extending element 350b.

The distal extending elements 350a, 350b extend parallel to the first elongate element 302. At its distal end 352a, the first distal extending element 350a is provided with the first clamp element 354 of the medial paddle. At its distal end 352b, the second distal extending element 350b is provided with the second clamp element 356 of the lateral paddle, as shown in FIG. 3e.

A variety of first clamp element 354 and second clamp element 356 designs can be employed in the instrument 300. This is possible without altering the rest of the instrument 300, if desired, potentially through interchangeable clamp elements.

In the design illustrated in FIGS. 3a-3e, the first clamp element 354 and second clamp element 356 are mirror images of one another. Each has a posterior extending element 358 which is generally elongate and of a lower profile than the posterior end portion 360. The posterior end portion 360 provides the clamping location 362 on an opposing surface 364 in each case.

Referring to FIGS. 3b and 3c, the opposing surface 364 is recessed within the posterior end portion 360, with the extent of recessing increasing from the posterior end 360 towards the anterior end 366 to a maximum depth 368 and then decreasing with further travel towards the anterior end 366. In a similar manner, FIG. 3a, the extent of recessing increases with travel from the proximal side 370 towards the distal side 372, through the maximum depth 368, and then decreasing with further travel towards the distal side 372. In effect a dished recess 374 is provided with the clamping location 362 within the recess 374. The overall structure of the first clamping element 354 and of the second clamping element 356 is similar to a spoon.

As shown in FIGS. 1b, 3b and 3c, the anatomy of many patients is such that the extremity M^{ext} of the medial malleolus 25 and the extremity L^{ext} of the lateral malleolus 31 are aligned with one another along direction 378, the same general direction as the separation distance 376 of the clamping elements, potentially both proximally-distally and anteriorly-posteriorly. To accommodate patients where this is not the case, further adjustability can be provided by the instrument.

For proximal-distal adjustment, it is possible to provide for extension and/or retraction of the first distal extending element 350a relative to the first offset element 342a. Telescoping of a first section of the first distal extending element 350a relative to a second section can be provided for this purpose, with a clamp or the like for fixing the positions. In this way different proximal-distal positions can be provided for the first distal extending element 350a and hence for the first clamp element 354 relative to the second distal extending element 350b and its clamping element 356. Even more flexibility can be provided by also allowing the second distal extending element 350a to also extend and/or retract relative to the second offset element 342b. Again, telescoping of a first section of the first distal extending element 350b relative to a second section can be provided for this purpose, with a clamp or the like for fixing the positions. Whilst extending and/or retracting the distal extending elements 350a, 350b extend parallel to the first elongate element 302 and equidistant from the first elongate element 302 such that the mid-point 305 between the paddle 301, 303 remains constant relative to the element 302 (and the proximal mounting element 412). Rotation is to be avoided to maintain correct alignments and so anti-rotation features, such as square cross-section elements can be used.

For anterior-posterior adjustment, it is possible to provide for an adjustable first clamping element 354, with alternatively or additionally, an adjustable second clamp element 356 provided. Again, a telescoping arrangement is an option, with a first section of the clamping element 354, 356 being received within a second section for this purpose. A further clamp can be used for fixing the positions.

In a similar manner, the first clamping element 354 may be pivotally mounted, with alternatively or additionally, the second clamp element 356 also pivotally mounted. The pivoting would be in the proximal-distal direction only and rotation in other directions is to be avoided to maintain correct alignments and so anti-rotation features, such as square cross-section elements can be used.

Referring again to FIG. 3a, at the proximal end 400 of the elongate element 302 is mounted a second elongate element 402 which in use extends in the posterior/anterior directions. The length of this second elongate element 402 in those directions can be adjusted as the second elongate element 402 is also formed of a first unit 404 and a second unit 406 seated within the first unit 404 and able to telescope into and out of the first unit 404. A further releasable clamp 408 is provided to fix the length at the desired extent. Typically, the telescopic arrangement seeks to provide a second variable length range of around 40 mm to 100 mm to be provided.

The ability to adjust the length of the second elongate element 402 is useful. To ensure correlation between the length selected for the second elongate element 402 with the first clamping element 354 and second clamping element 356 lengths, when those are variable in length, the second elongate element 402 can be provided with a scale such that the surgeon can obtained the length value. A similar scale and value set relating to the first clamping element 354 and second clamping element 356 can be used to ensure they are set at the matching value to ensure correct alignment.

At the posterior end 410 of the second elongate element 402, there is a mounting element 412 for a tibial engagement device. In this case, the tibial engagement element is in the form of a distal extending spike 414 configured to engage the ACL tibial insertion footprint 15. As shown in FIG. 3a, the spike 414 cooperates with the mid-point 305 defined between the paddles 301, 303 to define a longitudinal axis 307, which is positioned to be co-linear with the tibial mechanical axis P-D of the patient during use, thereby properly positioning the surgical instrument 300 for the proximal resection.

The mounting element 412 has a first position spaced from the proximal surface of the tibia 1 and a second position in which the spacing from the proximal surface of the tibia is reduced. The first and second positions and the spacing may be controlled by increasing or decreasing the length of the elongate element 302, in effect extending or contracting the elongate element 302 proximally-distally. At the desired position, the releasable clamp 308 may be fixed to maintain the position. In the second position, a tibial engagement element at least abuts and may penetrate the proximal surface of the tibia 1. In the case of spike 414 penetration is provided.

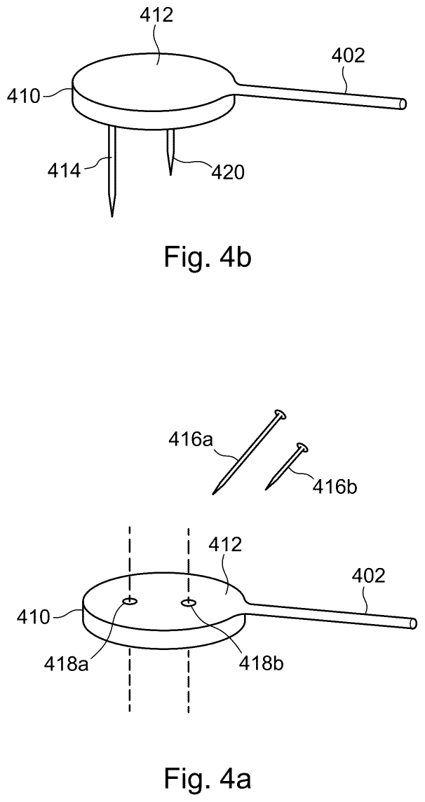

In the FIG. 4a embodiment of the instrument, the posterior end 410 of the second elongate element 402 again has a mounting element 412 for a tibial engagement device. In this case, the tibial engagement device is in the form of one or more pins [in this case two pins 416a, 416b] which, in use, pass through at least one aperture [in this case apertures 418a, 418b], penetrate and engage with the bone. Again, the tibial engagement device is sized and shaped to be positioned over (and aligned with) the ACL tibial insertion footprint 15 such that the tibial engagement device cooperates with the mid-point 305 between the paddles 301, 303 to define the longitudinal axis 307.

In this embodiment, the mounting element 412 continues to have a first position spaced from the proximal surface of the tibia 1 and a second position in which the spacing from the proximal surface of the tibia is reduced. The first and second positions and the spacing may be controlled by increasing or decreasing the length of the elongate element 302, in effect extending or contracting the elongate element 302 proximally-distally. At the desired position, the releasable clamp 308 may be fixed to maintain the position.

In the FIG. 4b embodiment of the instrument, the posterior end 410 of the second elongate element 402 again has a mounting element 412 for a tibial engagement device. In this case, the tibial engagement device is in the form of a long distal extending spike 414 for positioning the instrument 300 and a second shorter distal extending spike 420 which engages the bone after the longer distal extending spike 414 and which serves to prevent rotation of the instrument 300 about the longer spike 414. The spikes 414, 420 may be tapped into the bone by the surgeon during positioning. The first and second positions and the spacing may be controlled by increasing or decreasing the length of the elongate element 302, in effect extending or contracting the elongate element 302 proximally-distally. At the desired position, the releasable clamp 308 may be fixed to maintain the position.

Returning to the FIG. 3a-3e embodiment, in FIG. 3d the instrument 300 is connected in use to an adjustment component 740 using a connection mechanism 742. The connection mechanism 742 is provided by a mounting collar 744 slidably mounted on the elongate element 302. The elongate element 302 also extends distally-proximally through a slot 746 provided between a first posterior member 748 and second anterior member 750. The mounting collar 744 and slot 746 allow the adjustment component 740 and the elongate element 302 to move relative to one another.

The mounting collar 744 provides a pin 780 that extends in a posterior-anterior direction and provides a pivotal mounting for a bracket element 756. This means that the whole adjustment component 740 can be rotated relative to the elongate element 302 about that posterior-anterior axis 782. This in turn allows rotation of the proximal end 764 of the frame 760. As the mounting element 762 provides the cutting block 800 in use, this means that the necessary Varus and Valgus adjustability can be provided to the cutting block 800 and hence the cutting provided. The slot 746 accommodates the pivoting of the adjustment component 740 relative to the elongate rod 302 and a scale 782a and 782b can be provided on either side 784a, 784b of the second anterior member 750 to show the level of adjustment.

The bracket element 756 also provides a pair of pins 752a, 752b that pass through bracket arms 754a, 754b provided by a bracket element 756 and also pass through apertures 758a, 758b in a frame 760. This results in the frame 760 being pivotally mounted relative to the bracket arms 754a, 754b and bracket element 756, as well as relative to the mounting collar 744 and hence also relative to the elongate element 302. This allows inclination of the frame 760 to be varied relative to the elongate element 302, particularly the mounting element 762 provided at the proximal end 764 of the frame 760. As the mounting element 762 provides the cutting block 800 in use, this means that the slope of the cutting block relative to the tibia 1 can be controlled and hence the desired tibial slope can be applied in the subsequent cutting.

The cutting block 800 is mounted by a pair of posts, male elements 766 of the mounting element 762 engaging in use with a pair of holes, female elements 802 of the cutting block 800.

A plurality of different cutting blocks 800 with different slope angles can be provided, with the desired slope angle being determined and then the applicable cutting block 800 selected for connection and use. Such an approach could allow for an instrument design with less inbuilt adjustments required, for instance without the tibial slope adjustment described immediately above. The cutting blocks 800 could also have the Varus/Valgus adjustment built in so eliminating the need for adjustment in other components.

Returning to FIG. 3d, the frame 760 is supported as it pivots by a projection 768 extending from the second anterior member 750. The distal surface 770 of the projection 768 is defined by an a arc matching [plus a tolerance] with the arc the distal end 772 of the frame 760 moves through. One of both sides 786a, 786b of the projection 768 are provided with a scale 788a to show the level of adjustment. The ability to lock or ratchet both the frame 760 and the first posterior member 748 can also be provided.

In the surgical procedure, referring to FIG. 1a, the proximal end 5 of the tibia 1 is exposed. Between the first curved articular surface 7 and a second curved articular surface 9 there is the raised area formed of the medial intercondylar tubercule 11, the lateral intercondylar tubercule 13 and a notch 15 there between. This is the intended location for engagement with the spike 414.

As the surgical site is at the knee joint, whilst the ankle area is accessible, no surgical sites are generated there. The clamping on the patient is provided with the skin over the malleolus 25, 31 [the skin and tissue is omitted in the Figures for clarity and simplicity].

As a precursor to the steps involving the use of the instrument 300 of the disclosure, the following general steps may have occurred [not necessarily in this order and not necessarily any given step]:
Incision and exposure of the knee joint;
Femoral jig assembly and surgical parameter configuration;
Femoral jig insertion and fixing;
Femoral resection-typically distal femoral resection;
Femoral jig removal;
Femoral cutting block removal.

The stages involving the use of the instrument 300 of the disclosure then come into play.

In FIG. 3b, the instrument 300 is in the open state and the separation distance 376 between the posterior end portions 360 is greater than the medial to lateral extent 378 of any ankle joint 380. This allows the clamping elements 354, 356 to be advanced posteriorly with one clamping element to one side 382 of the ankle joint 380 and the other clamping element on the other side 384 of the ankle joint 380. With the recesses 374 generally aligned with the position of the medial malleolus 25 and the lateral malleolus 31, the transition from the open state towards the closed state can commence.

Movement of the collar element 310 causes the clamp operating mechanism 312 to reduce the separation distance 376. In practice, the clamp operating mechanism 312 may be biased to encourage the first clamping element 354 and the second clamping element 356 towards one another. For instance, the collar element 310 and the boss element 318 may abut a compression spring within the elongate element 302 between the collar element 310 and the boss element 318. The compression spring thus urges the collar element 310 and the boss element 318 apart and so urges a reduction in the separation distance 376 between the first clamping element 354 and the second clamping element 356. The surgeon can control the position of the collar element 310 on the elongate element 302 and hence the separation distance 376.

As the separation distance 376 decreases further the medial malleolus 25 and the lateral malleolus 31 start to enter their respective recesses 374. A further reduction in the separation distance 376 brings this down to the level of the medial to lateral extent 378 of the ankle joint 380, here the extremity M^{ext} of the medial malleolus 25 abuts the clamping location 362 and the extremity L^{ext} of the lateral malleolus 31 abuts the clamping location 362; the clamping state is reached.

As the separation distance 376 is varied, it is beneficial that the clamp operating mechanism 312 keeps the first clamping element 354 and the second clamping element 356 parallel with one another. As described above, the clamping mechanism operates to keep the first clamping element 354 and the second clamping element 356 equidistant from the first elongate element 302 such that the mid-point 305 between the paddle 301, 303 remains constant relative to the element 302 (and the proximal mounting element 412).

Thus, in the disclosure, the malleolus is received within the recess 374 and the extremity of the malleolus abuts the clamping location 362. The transition to this state, the clamping state, is shown in FIG. 3b moving to FIG. 3c and gives direct contact between the instrument 300 and the malleolus rather than the prior art clamping around the shin or around, but not contacting the malleolus.

With the clamping engagement at the ankle already provided, the surgeon can adjust the length of the first elongate element 302 to give the desired proximal/distal extent for the instrument 300. The surgeon can also adjust the length of the second elongate element 402 to move the mounting element 412 and distal extending spike 414 into position over the ACL tibial insertion footprint 15, which may be a notch, and, in an anterior-posterior direction, midway across the tibia 1. In effect this is at the central intersection of the coronal plane and the sagittal plane of the tibia 1. In this position, the further releasable clamp 408 is used to fix the extent of the second elongate element. The spike 414 is engaged with the proximal surface of the tibia 3 by the surgeon. The mounting element 412 may be tapped to drive the spike 414 into a firm engagement in this position.

It is possible to mount the proximal end 400 first and then clamp the distal ankle end second, if preferred.

With the instrument 300 firmly and accurately clamped in position at the ankle and at the knee, the longitudinal axis 307 is positioned to be co-linear with the tibial mechanical axis P-D of the patient during use, thereby properly positioning the surgical instrument 300 for the proximal resection. The surgeon can then check the alignment and if correct proceed with the subsequent steps of the surgery. The alignment check would seek to ensure that the elongate element 302 is parallel to the tibial axis viewed laterally-medially and still aligned with the tibial mechanical axis P-D when viewed posteriorly.

After the alignment check, as an initial step, if not already mounted on the instrument 300, then the adjustment component 740 is introduced and connected using the connection mechanism described above.

Also, if not already present, a tibial cutting block 800 is chosen and connected to the adjustment component 740 using the cutting block connection mechanism described above.

Using the adjustment component 740, and rotating it relative to the elongate member 302 in a medial-lateral direction, the desired Valgus or Varus adjustment can be provided for the cutting block 800. Again using the adjustment component 740 and rotating it relative to the elongate element 302 in a posterior-anterior direction, the desired tibial posterior slope can be set.

Once all alignment and orientation variables have been set to the intended values, then a stylus attachment is connected to the cutting block and used to set the resection amount and the height. The tibial cutting block 800 can then be pinned to the bone and the tibial resection can be provided.

After the resection of the tibia, the removal of the instrument 300 and adjustment component 700 can be conducted, with the following general steps following in the surgical procedure [not necessarily in this order and not necessarily any given step]:
Extension gap assessment and balancing, for instance using spacer blocks;
Femoral rotation, for instance using a measured sizer or balanced sizer—including introduction of the sizer, pinning and removal as potential steps;
Balanced femoral sizing and rotation, for instance to size the femur, set the rotation for the femoral component and assess the flexion gap;
Femoral preparation, for instance provision of the chamfer block, pinning, flexion space checking, cutting and removal;
Posterior condyle preparation, including removal of excess bone, sulcus femoral preparation;
Femoral resection, including notch cutting;
Trial reduction;
Trial component selection and use;
Tibial preparation, including drilling, keel punching;
Patella resection and preparation;
Lug hole preparation;
Tibial base implantation;
Femoral component implantation;
Tibial insert implantation;
Final patella preparation;
Patella component implantation;
Closure.

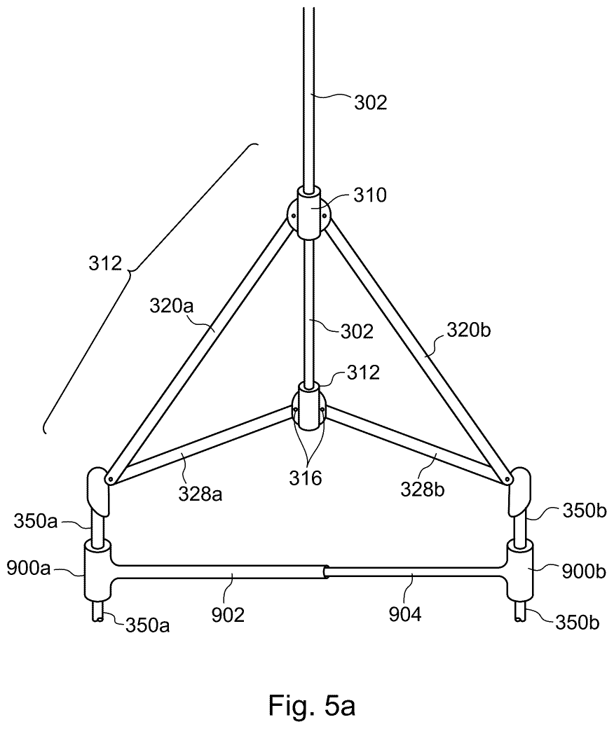

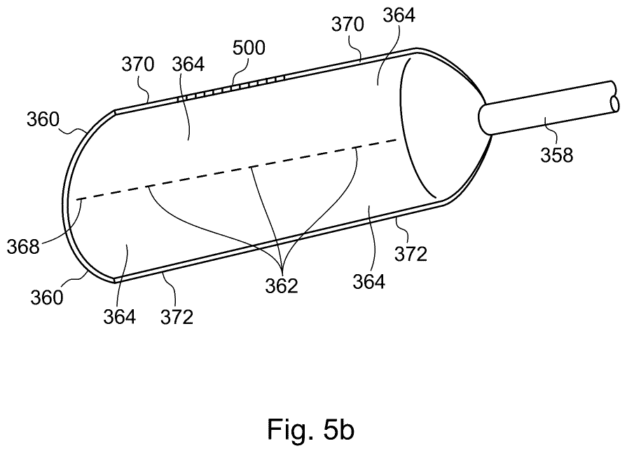

Returning to the instrument 300 and its design, other forms for the clamp operating mechanism 312 can be provided. In the FIG. 5a embodiment of the instrument, the second pivotal mounting 316 provided on a boss element 318 is still used, but the distal arms, the second pair of operating arms 328a, 328b are inclined in the same general direction [proximally] as the first pair of operating arms 320a, 320b, the proximal arms. This means that the distal arms and section of the clamp operating mechanism extends less in the distal direction and so less operating space is required. This is the general location of the patient's foot, in use. The pivots are still provided in the same manner and the ability of the collar element 310 to slide proximally and distally on the elongate element 302 increases and decreases the separation distance 376.

In the FIG. 5b embodiment, an alternative form for the first clamp element 354 and second clamp element 356 is provided. Again, both are mirror images of one another. Each has a posterior extending element 358 which is generally elongate and of a lower profile than the posterior end portion 360. The posterior end portion 360 provides the clamping location 362 on an opposing surface 364 in each case. In this case, however, the opposing surface 364 is of a different profile. Once again, the extent of recessing increases with travel from the proximal side 370 towards the distal side 372, through the maximum depth 368, and then decreasing with further travel towards the distal side 372. However, from the posterior end portion 360, along any given anterior-posterior extending line, the extent of recessing is consistent from the posterior end towards the anterior end 366. Thus, the maximum depth 368 is present along the length of an anterior-posterior line A-P substantially on the mid-line of the opposing surface. In effect a C-shaped cross-section recess 374 is provided which is more open at both the posterior end and the anterior end.

In use, once again the extremity M^{ext} of the medial malleolus 25 and the extremity L^{ext} of the lateral malleolus 31 are aligned with and will engage with the clamping location 362. However, as the anterior-posterior position of the clamping location 362 is not as defined as with the earlier embodiment, a scale 500 is provided extending along the proximal edge of the posterior end portion 360 so that the surgeon can establish the alignment of the clamping location 362 with a value for the scale 500 and the same value can be used in the setting of the anterior-posterior extent of the second elongate element 402 which in use extends in the posterior/anterior directions to and provides the spike 414. Thus, correctly configured anterior-posterior positions are provided at both the distal and the proximal ends of the instrument 300 and the correct alignment is provided with the tibia.

In terms of anatomy, most patients are consistent enough that the tibial mechanical axis P-D can be defined at a mid-point relative to the two malleolus limits. This can be 50:50 in terms of the chosen mid-points. As some surgical approaches prefer the use of a 55:45 split on the setting of the tibial mechanical axis P-D, embodiments of the instrument can readily be provided configured to accommodate that split. This choice is generally one of surgical concept, rather than individual patient anatomy and so typically a surgeon will have a given embodiment of instrument rather than pick between them for a given patient, but that is an option within the disclosure.

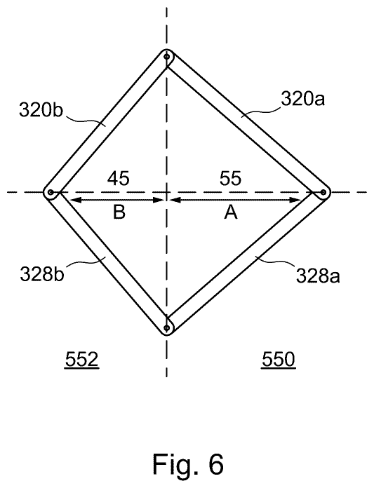

To change the split, as shown in FIG. 6, the revised embodiment uses a first side first operating arm 320a and a first side second operating arm 328a which are of a different length to the second side first operating arm 320b and a second side second operating arm 328b. In this case, the first side 550 are longer than the second side 552. The lengths are selected to give 55% of the movement inward and outward on the first side [distance A] and 45% on the second side [distance B]. These distances directly correspond to the movement of the first clamping element 354 and the second clamping element 356 and hence of the position of the elongate element 302 relative to the extremity M^{ext} of the medial malleolus 25 and the extremity L^{ext} of the lateral malleolus 31.

To keep the first distal extending element 350a aligned in the same distal-proximal direction, as the arms 326a, 328a move, and to keep the first distal extending element parallel to the second distal extending element 350b, FIG. 5a includes a different form of anti-rotation approach to that in FIGS. 3a and 3e. The approach is useful for all instrument, but particularly so where the different arm pairs 320a, 328a as opposed to arm pair 320b, 328b move through different angles due to their different lengths; see FIG. 6 described above, particularly if arranged in the FIG. 5a orientation.

In the FIG. 5a approach, the first distal extending element 350a is provided with a brace element 900a in the form of an annular collar extending distally-proximally. The second distal extending element 350b is also provided with a brace element 900b. A first stem 902 extend from the brace element 900a and is hollow to receive a telescoping second stem, 904 extending from the brace element 900b. The telescoping of the stems 902, 904 allows the arms 320a, 320b, 328a, 328b to move and reduce the separation between the first distally extending element 350a and the second distally extending element 350b, however, the stems 902, 904 and brace elements 900a, 900b resist any rotation and so keep the first distal extending element 350a parallel to the second distal extending element 350b.

Whilst with an embodiment like that of FIG. 3a-3c, the instrument can be switched between right and left legs readily [as the 50:50 centre split applies], for embodiments like FIG. 6 where the 55:45 split is employed then the clamp operating mechanism 312 needs to be rotated through 180° and so the first clamping element 354 and the second clamping element 356 need to be rotatably mounted on the first and second distal extending elements to provide them in the correction orientation to be put around the other side ankle.

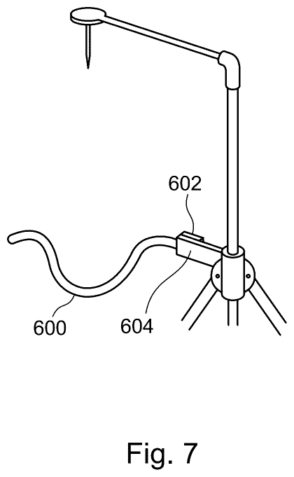

Whilst spring loaded biasing of the clamps and the tap in spike 414 are sufficient to position and maintain that position for the instrument 300, it may be useful to provide a secondary clamping function too. Referring to the FIG. 7 embodiment, this is substantially the same as the FIG. 3a-3c embodiment, but in addition it includes a strap 600 which extends from one side of the instrument 300 and can be wrapped around the leg of the patient and tightened using catch 602 on a mounting 604 the other side of the instrument 300. This secondary clamping function assists in giving firm retention of the instrument 300 in position once the surgeon has established the datum of the tibia. Various secondary clamping and various positions for this could be provided, for instance around the ankle or foot or higher on the tibia/fibula. The strap and catch could be replaced or supplemented by one or more springs or sprung elements that extend around the leg before fastening.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. A surgical instrument, the instrument comprising:
an elongate element having a distal portion and a proximal portion and extending in a proximal-distal direction;
a clamp operating mechanism mounted on the elongate element;
two or more clamp elements, connected to the distal portion of the elongate element and controlled by the clamp operating mechanism, the two or more clamp elements having a first open state and a second engaging state, wherein (i) the clamp operating mechanism is operable to move the two or more clamp elements between the first open state and the second engaging state, and (ii) the two or more clamp elements have a mid-point that remains constant between the first open state and the second engaging state; and
a mounting element, connected to the proximal portion of the elongate element, wherein (i) the mounting element has a first position in the proximal-distal direction and a second position in the proximal-distal direction, the first position being further from the two or more clamp elements than the second position, and (ii) the mounting element and the mid-point between the clamp elements define a longitudinal axis that is configured to be positioned co-linear with a tibial mechanical axis of a patient during use.

2. The surgical instrument of claim 1, wherein one or more of the clamping elements in the engaging state, in use, is configured to engage with a malleolus of the patient.

3. The surgical instrument of claim 1, wherein one or more of the clamping elements in the engaging state, in use, is configured to engage with a medial malleolus of the tibia and a lateral malleolus of the fibula of the patient.

4. The surgical instrument of claim 1, wherein, in use, the mounting element, and/or a depending element extending therefrom, in the first position is configured to oppose a portion of an ACL tibial insertion footprint of a tibia.

5. The surgical instrument of claim 1, wherein;
one or more of the clamping elements in the engaging state, in use, is configured to engage with a medial extremity of the medial malleolus, and
one or more of the clamping elements in the engaging state, in use, is configured to engage with a lateral extremity of the lateral malleolus of the patient.

6. The surgical instrument of claim 1, wherein two or more clamping elements in the engaging state, in use, are configured to engage with a medial extremity of the medial malleolus of the tibia and a lateral extremity of the lateral malleolus of the fibula of the patient.

7. The surgical instrument of claim 1, wherein:
the elongate element has a variable proximal-distal direction length,
a greater proximal-distal extent is provided in the first position for the mounting element, and
a lesser proximal-distal extent is provided in the second position for the mounting element.

8. The surgical instrument of claim 1, wherein:
in use, the mounting element, and/or a depending element extending therefrom, is configured to be proximally spaced from a first anatomical feature of the patient in the first position, and
in use, the depending element extending therefrom is configured to penetrate the first anatomical feature of the patient in the second position.

9. The surgical instrument of claim 1, wherein:
the mounting element has (i) a plurality of depending elements extending therefrom, and (ii) a plurality of spikes and/or a plurality of pins.

10. The surgical instrument of claim 1, wherein the first open state for the two or more clamping elements, in use, is configured to allow one or more clamp elements to be introduced to one side of a second anatomical feature of the patient and one or more other clamp elements to be introduced to a second side of a further anatomical feature of the patient.

11. The surgical instrument of claim 1, wherein the second engaging state for the two or more clamping elements, in use, is configured to prevent one or more clamp elements being removed from one side of an anatomical feature of the patient and also configured to prevent one or more other clamp elements being removed from a second side of an anatomical feature of the patient.

12. (canceled)

13. The surgical instrument of claim 1, wherein:
one or more or all of the clamping elements include a posterior extending element with a posterior end portion,
the posterior end portion defines a surface recessed within the posterior end portion, and
the recessed surface including a clamping location.

14. The surgical instrument of claim 1, wherein the elongate element has a variable proximal-distal direction length.

15. The surgical instrument of claim 1, wherein;
the mounting element is connected to the proximal portion of the elongate element by a second elongate element, and
the second elongate element extends in the anterior-posterior direction and has a variable anterior-posterior length.

16. The surgical instrument of claim 1, wherein one or more or all of the clamping elements extend in the anterior-posterior direction and one or more or all of the clamping elements have a variable anterior-posterior length.

17. A surgical instrument system, the instrument comprising:
a first surgical instrument,
a second surgical instrument for releasable connection to the first instrument;
the first surgical instrument comprising:
an elongate element having a distal portion and a proximal portion and extending in a proximal-distal direction;
a clamp operating mechanism mounted on the elongate element;
two or more clamp elements, connected to the distal portion of the elongate element and controlled by the clamp operating mechanism, the two or more clamp elements having a first open state and a second engaging state, wherein (i) the clamp operating mechanism is operable to move the two or more clamp elements between the first open state and the second engaging state, and (ii) the two or more clamp elements have a mid-point that remains constant between the first open state and the second engaging state; and;
a mounting element, connected to the proximal portion of the elongate element, wherein (i) the mounting element has a first position in the proximal-distal direction and a second position in the proximal-distal direction, the first position being further from the two or more clamp elements than the second position, and (ii) the mounting element and the mid-point between the clamp elements define a longitudinal axis that is configured to be positioned co-linear with a tibial mechanical axis of a patient during use.

18. (canceled)

19. The surgical instrument system of claim 17, wherein the second surgical instrument is an adjustment component for mounting on the first instrument.

20. The surgical instrument system of claim 19, wherein the adjustment component includes a first pivotal mounting providing rotation of the adjustment component relative to the first instrument in a medial-lateral direction

21. The surgical instrument system of claim 17, wherein the adjustment component includes a second pivotal mounting providing rotation of the adjustment component relative to the first instrument in an anterior-posterior direction.

22. The surgical instrument system of claim 17, wherein the system includes a third instrument, the third instrument being one or more cutting blocks.

23. A surgical method, the method comprising:
providing an instrument, the instrument comprising:
an elongate element having a distal portion and a proximal portion and extending in a proximal-distal direction;
a clamp operating mechanism mounted on the elongate element;
two or more clamp elements, connected to the distal portion of the elongate element and controlled by the clamp operating mechanism, the two or more clamp elements having a first open state, a second engaging state, and;
a mounting element, connected to the proximal portion of the elongate element, the mounting element having a first position in the proximal-distal direction and a second position in the proximal-distal direction, the first position being further from the two or more clamp elements than the second position;
providing the two or more clamping elements in a first open state;
exposing the proximal end of the tibia of a patient at a surgical site;
abutting the mounting element or an element depending therefrom with the proximal end of the tibia within the surgical site;
introducing the two or more clamping elements to the ankle region of the patient;
transitioning the two or more clamping elements from the first open state to the second engaging state to engage the clamping elements with the ankle region of the patient such that the mounting element and the mid-point between the clamp elements define a longitudinal axis that is co-linear with a tibial mechanical axis of the patient.

24. (canceled)

25. (canceled)
